(19) United States
(12) Patent Application Publication
(10) Pub. No.: US 2015/0375008 A1
(43) Pub. Date: Dec. 31, 2015

(54) PORTABLE ILLUMINATING DEVICE FOR MANIPULATING THE CIRCADIAN CLOCK OF A SUBJECT

(71) Applicants: Norbert GRETZ, Mannheim (DE); Yury SHULHEVICH, Mannheim (DE)

(72) Inventors: Norbert GRETZ, Mannheim (DE); Yury SHULHEVICH, Mannheim (DE)

(21) Appl. No.: 14/767,998

(22) PCT Filed: Feb. 20, 2014
(86) PCT No.: PCT/EP2014/053286

§ 371 Date: Aug. 14, 2015

(30) Foreign Application Priority Data
Feb. 20, 2013 (EP) 13155946.0

(51) Int. Cl. A61N 5/06; H05B 33/08

(52) U.S. Cl. CPC A61N 5/0618; H05B 33/0854; A61N 2005/0648; A61N 2005/0652; A61N 2005/0653; A61N 2005/0662; A61N 2005/0666

(57) ABSTRACT

The present invention relates to a portable illuminating device (110) comprising at least one illuminating light source (112) emitting light of a wavelength comprised in the range of 450 to 495 nm, preferably, comprising at least one further illuminating light source (112) emitting light of a wavelength comprised in the range of 620 nm to 750 nm; a diffuser (113) scattering the light of said illuminating light source (112) at least in part in the direction of at least one eye, preferably at least one cornea and/or retina, of said subject; and a mounting mechanism (114) for fixing the portable illuminating device (110) to the body of a subject. Preferably, said diffuser (113) scatters the light of said illuminating light source (112) at least in part in the direction of at least one eye and in the direction of at least a part of the facial skin of a subject. Preferably, the portable illuminating device (110) further comprises a sensor for determining if illumination is advisable, preferably a melatonin sensor, and/or comprises an oxygen and/or pulse sensor. The present invention further relates to the portable illuminating device (110) for use in preventing and/or treating a disease or disorder caused by or associated with a distorted circadian rhythm; and to a method of improving physical and/or mental performance of a healthy subject, comprising the step of illuminating at least one cornea of said subject with light comprising light of a wavelength of 450 to 495 nm for at least 15 min, thereby improving physical and/or mental performance of a healthy subject. Preferably, the invention relates to a method of improving physical and/or mental performance of a healthy subject, comprising illuminating at least one eye and/or at least a part of the facial skin of said subject with light comprising light of a wavelength of 450 nm to 495 nm and/or illuminating said subject with light comprising light of a wavelength of 620 nm to 750 nm, thereby improving physical and/or mental performance of a healthy subject.

The present invention relates to a portable illuminating device comprising at least one illuminating light source emitting light of a wavelength comprised in the range of 450 to 495 nm, preferably, comprising at least one further illuminating light source emitting light of a wavelength comprised in the range of 620 nm to 750 nm; a diffuser scattering the light of said illuminating light source at least in part in the direction of at least one eye, preferably at least one cornea and/or retina, of said subject; and a mounting mechanism for fixing the portable illuminating device to the body of a subject. Preferably, said diffuser scatters the light of said illuminating light source at least in part in the direction of at least one eye and in the direction of at least a part of the facial skin of a subject. Preferably, the portable illuminating device further comprises a sensor for determining if illumination is advisable, preferably a melatonin sensor, and/or comprises an oxygen and/or pulse sensor. The present invention further relates to the portable illuminating device for use in preventing and/or treating a disease or disorder caused by or associated with a distorted circadian rhythm; and to a method of improving physical and/or mental performance of a healthy subject, comprising the step of illuminating at least one cornea of said subject with light comprising light of a wavelength of 450 to 495 nm for at least 15 min, thereby improving physical and/or mental performance of a healthy subject. Preferably, the invention relates to a method of improving physical and/or mental performance of a healthy subject, comprising illuminating at least one eye and/or at least a part of the facial skin of said subject with light comprising light of a wavelength of 450 nm to 495 nm and/or illuminating said subject with light comprising light of a wavelength of 620 nm to 750 nm, thereby improving physical and/or mental performance of a healthy subject.

Circadian rhythms are known as biological processes showing an oscillation of about one day, governing basic biological processes such as the sleep-wake cycle of mammals. It has been known for some time that circadian rhythms are, in principle, endogenously controlled; however, they are entrained, i.e. readjusted, by external zeitgebers such as ambient light. Disruption of the normal circadian rhythm can have several causes, the most frequent being travel over several time zones and shift-work. The condition associated with a circadian rhythm out of sync with local time after time zone travel is referred to as Rapid Time Zone Change Syndrome or desynchronosis, commonly known as “jet-lag”, typical symptoms being sleep disturbances (difficulty of falling asleep and/or awakening, interrupted sleep), cognitive effects like fatigue, drowsiness, headache, irritability, but also including e.g. digestion problems (Reilly et al. (2007); Eur J Sport Sci 7(1): 1-7). The major hormone known to induce the physiological changes associated with cicadian rhythms is melatonin, which is produced by the epiphysis. Interestingly, it was found that also skin cells are able to synthesize melatonin, which led to speculations on the possible role of melatonin in the regulation of skin functions (Moreno et al. (2013), FEBS J 280:4782).

The primary, endogenous clock in mammals is located in the hypothalamus, specifically in the suprachiasmatic nucleus. Entrainment by ambient light is mediated by a subset of intrinsically photosensitive retinal ganglion cells (ipRGCs) expressing the photopigment melanopsin, which is sensitive to blue light; it has, however, also been shown that entrainment can at least in part be mediated by cones sensitive to green light. ipRGCs are present at low density over the whole retina and receive direct input from rod and cones, which is believed to explain the additive effect of green light (Gooley et al. (2010), Sci Transl Med 2, 31ra33: 1-9). Signaling by ipRGCs induced by light perception suppresses formation of melatonin, the major hormone governing wake-sleep cycles, in the epiphysis. In addition, other principal bodily functions, such as alertness, thermoregulation, and heart rate, are influenced by light (Cajochen et al. (2005), J Clin Endocrinol Metab 90(3): 1311-1316), a fact which also was used to increase physical performance of healthy subjects (Kantermann et al. (2012), PLOS one 7(7): e40655). Only recently, it was found that cells of the skin, in particular of the epidermis, which consists mainly of keratinocytes and melanocytes, also have photoreceptors and change gene expression patterns following illumination. Apart from potentially influencing the differentiation state of keratinocytes, no physiological role of photoreceptor-like proteins in human dermis could be assigned (Kim et al. (2013), PLOS One 8(9):e73678).

It has been proposed earlier that bright light, especially blue light, might be used to assist in re-syncing the circadian clock of an individual with local time. Depending on the latitudinal direction of travel, specific lighting schemes have been proposed. However, the lighting schemes proposed rely on an improved timing of ambient lighting, i.e. of spending time outside or in a room illuminated with bright light. This, however, makes e.g. re-syncing the circadian clock of a single individual living among other individuals difficult. Moreover, a device has been provided for irradiation of a subject with blue light (Philips goLITE BLU, Revell et al. (2012), J Physiol 590.19: 4859-4868); however, the device is designed as a desktop appliance, and thus is mainly useful if short illumination cycles are performed while the subject is seated close to the device.

There is, thus, a need in the art to provide reliable means and methods to re-sync the circadian clock of an individual with local time. This problem is solved by the present invention and by the embodiments described herein.

Accordingly, the present invention relates to a portable illuminating device comprising:
i) at least one illuminating light source emitting light of a wavelength comprised in the range of 450 to 495 nm;
ii) a diffuser scattering the light of said illuminating light source at least in part in the direction of at least one eye, preferably at least one cornea and/or retina, of a subject; and
iii) a mounting mechanism for fixing the portable device to the body of said subject.

As used herein and in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B”, and “A includes B” may all refer to a situation in which, besides B, no other element is present in A (i.e. a situation in which A solely and exclusively consists of B) and to a situation in which, besides B, one or more further elements are present in entity A, such as element C, elements C and D, or even further elements.

As used herein, the term “subject” relates to an animal, preferably an animal having a circadian rhythm, more preferably an animal having a circadian rhythm influenced, most preferably controllable, by blue light. Preferably, the subject is a subject having a circadian variation in the concentration of at least one metabolite influenced, preferably controllable, by blue light. Preferably, said metabolite is a hormone, more preferably a hormone affecting the day-night-cycle of said animal, most preferably the metabolite is melatonin. In another preferred embodiment, the metabolite influenced, preferably controllable, by blue light is a steroid. Preferably, the subject is a mammal, more preferably a companion animal like, e.g., a dog, a cat, a hamster, a guinea pig, a rabbit, a hare, a horse, a cow, or a pig. Most preferably, the subject is a human.

The terms “eye”, “cornea”, and “retina” are anatomical terms and are used in their usual meaning known in the art. The term “body” relates to the complete body of a subject or to parts thereof. Preferably, body relates to a body part close to at least one eye of a subject, more preferably to the head, forehead, ear, and/or nose of a subject.

The term “light source”, as used herein, relates to any means capable of emitting photons of at least one wavelength as specified herein below. Preferably, the light source is a lamp, a light emitting diode (LED), or an organic light emitting diode (OLED). Preferably, the light source emits a spectrum of photons comprising photons having the wavelength as specified herein; i.e. the light source emits light of various wavelengths, at least one of said wavelengths being comprised in the spectrum as specified herein. More preferably, the light source emits a spectrum of photons essentially consisting of photons having the wavelengths as specified herein; i.e. the light source essentially emits light of wavelengths as specified. Most preferably, the light source emits a spectrum of photons consisting of photons having the wavelengths as specified herein; i.e. the light source emits light of wavelengths as specified. It is, however, also envisaged by the present invention that the light source emits photons of one wavelength comprised by the wavelengths as specified. In a preferred embodiment, it is envisaged that the light source emits photons of one or more discrete wavelengths, of which at least one, preferably all, are comprised by the wavelengths as specified. Preferably, the light source comprises a plurality of photon-generating devices having the same or different spectral emission properties as described herein below. It is to be understood that some embodiments of the present invention can comprise two kinds of light source: the light source providing the light scattered by the diffuser at least in part in the direction of at least one eye of a subject, which is referred to as “illuminating light source”, and the radiation source, preferably light source, comprised in the sensor plaster as detailed herein below, which is referred to as “sensor radiation source” or as “sensor light source”. In a preferred embodiment, the light source emits light in pulse mode, e.g. in pulses of 1 or 2 s every minute. In a further preferred embodiment, the light source emits light in continuous mode.

At least one of the wavelengths of the light emitted by the illuminating light source of the present invention is comprised in the range 450 nm to 495 nm (blue light). Preferably, said range is 460 nm to 480 nm. Preferably, the wavelength of maximal intensity of the light emitted by the illuminating light source is in the range 450 nm to 480 nm, more preferably 465 nm to 475 nm. Preferably, at least one photon-generating device comprised in the illuminating light source is a blue LED or a blue OLED. The skilled person understands that light of a wavelength of less than 450 nm may potentially harm the eye; thus, preferably, the illuminating light source does not emit light of a wavelength of less than 450 nm or essentially does not emit light of a wavelength of less than 450 nm.

In a preferred embodiment, the illuminating light source of the present invention additionally or alternatively emits light comprised in the range 495 nm to 600 nm (green light), preferably 500 nm to 590 nm, more preferably 520 nm to 580 nm, most preferably 530 nm to 560 nm. Preferably, a wavelength of a second maximal intensity of the light emitted by the illuminating light source is in the range 530 nm to 580 nm, more preferably 545 nm to 565 nm, most preferably about 555 nm. Thus, preferably, at least a second photon-generating device comprised in the illuminating light source is a green LED or a green OLED. It is understood by the skilled person that green light illumination induces at least partly the same effects on circadian rhythms as blue light illumination does. In particular, during the early time of illumination and/or low irradiances, the effects are similar (Gooley et al., loc cit.). Accordingly, preferably, the skilled person understands that, in particular under the aforementioned preconditions, green light may be used in addition to or replace blue light in to achieve the effects of the present invention.

In a further preferred embodiment, the illuminating light source of the present invention additionally emits light comprised in the range 600 nm to 800 nm (red light), preferably 605 nm to 700 nm, more preferably 610 nm to 650 nm, most preferably 610 nm to 620 nm or 620 nm to 645 nm. Preferably, a wavelength of another maximal intensity of the light emitted by the illuminating light source is in the range 600 nm to 800 nm, more preferably 605 nm to 700 nm, even more preferably 610 nm to 650 nm, most preferably 610 nm to 620 nm or 620 nm to 645 nm. Thus, preferably, at least a second photon-generating device comprised in the illuminating light source is a red-orange LED, a red LED, or a red OLED. In a further preferred embodiment, the illuminating light source of the present invention additionally or alternatively emits light comprised in the range 620 nm to 750 nm.

The portable illuminating device, preferably, comprises a plurality of photon-generating devices and/or illuminating light sources. More preferably, the plurality of photon-generating devices and/or illuminating light sources is arranged in alignment with a rim of the portable illuminating device, most preferably a rim of the diffuser. It is understood by the skilled person that a portable illuminating device according to the present invention comprising at least one photon-generating device emitting photons of blue light and at least one second photon-generating device emitting photons of green light can be operated in a blue light only mode, a green light only mode, in a blue light plus green light mode, and in a no light mode, i.e. switched off. Mutatis mutandis, a portable illuminating device according to the present invention comprising at least one photon-generating device emitting photons of blue and/or green light and at least a further photon-generating device emitting photons of red light can be operated in a blue or green light only mode, a red light only mode, in a blue light plus green light mode, and in a no light mode, i.e. switched off. It is understood that, in principle, a blue and green and red mode is also possible. Thus, the portable illuminating device of the present invention, preferably, comprises at least one switch for at least one of switching on or off the light, changing an intensity of the light, or changing the wavelength of the light.

In a preferred embodiment, the illuminating light source is comprised in a flexible matrix, preferably a gel, more preferably a washable matrix, most preferably a machine washable matrix. Preferably, said matrix is flexible, but is rigid enough to support, e.g. the bill of a baseball cap. In a further preferred embodiment, said matrix also comprises required circuitry for the illuminating light source. In an also preferred embodiment, the illuminating light source is connected to the other electronic elements of the portable illuminating device by a connector which can be used to detach said other electronic elements from the illuminating light source before washing. It is, however, also considered that, in a preferred embodiment, all electronic elements are attached to the portable illuminating device such that they can be removed before washing and re-mounted after washing.

Preferably, the illuminance provided by the portable illuminating device on a cornea of a subject is at least 10 lux (lm/m²), at least 20 lux (lm/m²), at least 30 lux (lm/m²), at least 40 lux (lm/m²), at least 50 lux (lm/m²), at least 60 lux (lm/m²), at least 70 lux (lm/m²), at least 80 lux (lm/m²), at least 90 lux (lm/m²), at least 100 lux (lm/m²), at least 125 lux (lm/m²), at least 150 lux (lm/m²), at least 200 lux (lm/m²), or at least 250 lux (lm/m²). More preferably, the illuminance of the light of a wavelength as specified above provided by the portable illuminating device on a cornea of a subject is at least 10 lux (lm/m²), at least 20 lux (lm/m²), at least 30 lux (lm/m²), at least 40 lux (lm/m²), at least 50 lux (lm/m²), at least 60 lux (lm/m²), at least 70 lux (lm/m²), at least 80 lux (lm/m²), at least 90 lux (lm/m²), at least 100 lux (lm/m²), at least 125 lux (lm/m²), at least 150 lux (lm/m²), at least 200 lux (lm/m²), or at least 250 lux (lm/m²).

The term “diffuser” relates to any means effecting scattering of light emitted by an illuminating light source. The skilled person knows that diffusion of light can be effected by diffuse reflection and/or by roughness of the surface of a diffuser, as, e.g. in frosted glass. The diffuser may comprise or be a rigid or, preferably, a flexible and/or adjustable structure. Preferably, the diffuser is mounted above or below the eyes of a subject, more preferably, particularly if the subject is a human, the diffuser is mounted at or close to the forehead of a subject. Preferably, the diffuser scatters light emitted by the illuminating light source at least in part in the direction of at least one eye of a subject. More preferably, the diffuser is shaped such as to effect preferred reflection in the direction of at least one eye of a subject, preferably, by a bent shape. In a preferred embodiment, the diffuser scatters light emitted by the illuminating light source at least in part in the direction of at least one eye and in the direction of at least a part of the facial skin of a subject. Preferably, the diffuser comprises one of a brim, preferably a curved brim, of a peaked cap, more preferably of a baseball cap, of a headband, or a sunblind of a headwear. It is, however, also envisaged that the diffusor, preferably, is a structure attached to the illuminating light source, more preferably a structure enclosing the illuminating light source at least in part, even more preferably a structure enclosing the parts of the illuminating light source emitting light in the direction of at least one eye of a subject, most preferably a structure at least covering those parts of the illuminating light source emitting light. It is understood by the skilled person that in case the diffuser covers the illuminating light source at least in part, the diffuser, preferably, is made of a translucent, but not clear-transparent material. Preferred materials are frosted variants of glass, acrylic glass, or plastic, or other appropriate materials like, e.g., silicone.

It is known to the skilled person that the site of action of blue light in the mammalian organism is the retina. Thus, it is understood that the term “reflecting in the direction of at least one eye” preferably relates to reflecting such that the light enters the eye and illuminates at least a portion of at least one retina of the subject. Preferably, both eyes and, thus, at least parts of both retinas are illuminated. Preferably, at least 50%, at least 60%, at least 70%, at least 80%, or at least 90% of the light illuminating at least part of at least one retina enters the eye in an angle relative to the viewing axis of 45° to 60° above the viewing axis, more preferably, at least 50%, at least 60%, at least 70%, at least 80%, or at least 90% of the light illuminating at least part of at least one retina enters the eye in an angle relative to the viewing axis of 0° to 45° above the viewing axis. It is known to the skilled person that blinding is aggravated with increasing intensity of illumination of the fovea, also known as fovea centralis. Thus, preferably, reflecting in the direction of at least one eye relates to reflecting such that at most 30%, at most 20%, at most 10%, at most 5%, at most 1%, at most 0.1% of the light energy illuminates the fovea.

As it was newly found in the work underlying the present invention, skin cells are a secondary site of action of visible light, preferably blue light. Accordingly, in a preferred embodiment, the diffuser scatters light emitted by the illuminating light source at least in part in the direction of at least one eye and in the direction of at least a part of the facial skin of a subject. In a preferred embodiment, the diffuser scatters light emitted by the illuminating light source such that at least 20%, preferably at least 30%, more preferably at least 40%, or, most preferably, at least 50% of the facial skin are illuminated; preferably, said fractions of the skin are illuminated with an average illuminance of at least 100 lux (lm/m²), at least 500 lux (lm/m²), at least 1000 lux (lm/m²), at least 5000 lux (lm/m²), or at least 10000 lux (lm/m²), wherein the average illuminance is preferably calculated as the average of the illuminated area; more preferably, said average is the average of measurements at five randomly selected points in said area. In an equally preferred embodiment, the diffuser scatters light emitted by the illuminating light source such that at most 20%, more preferably at most 10%, or, most preferably, at most 5% of the light scattered in the direction of at least one eye and in the direction of at least a part of the facial skin of the subject illuminates a retina or the retinas of said subject as described herein above, wherein the portion of light illuminating a retina or the retinas is preferably determined on a subject having its eyes open. As will be understood by the skilled person, the portion of light illuminating a retina or the retinas may be close to 0 or may be 0 in case the subject has its eyes closed. Accordingly, preferably, the diffuser scatters light emitted by the illuminating light source such that at least 80%, more preferably at least 90%, or, most preferably, at least 95% of the light scattered in the direction of at least one eye and in the direction of at least a part of the facial skin of the subject illuminates the facial skin. It is understood that the aforesaid embodiments can, preferably, be combined, e.g. to provide a diffuser, wherein said diffuser scatters light emitted by the illuminating light source such that at least 20%, preferably at least 30%, more preferably at least 40%, or, most preferably, at least 50% of the facial skin are illuminated, and wherein said diffuser scatters light emitted by the illuminating light source such that at most 10%, preferably at most 5%, more preferably at most 2%, or, most preferably, at most 1% of the light scattered at least in part in the direction of at least one eye and in the direction of at least a part of the facial skin of the subject illuminates a retina or the retinas of said subject.

In a preferred embodiment, the diffuser is located distal relative to the illuminating light source. In a more preferred embodiment, the diffuser is located proximal to the illuminating light source; it is understood by the skilled person that the diffuser will be of a material of an optical density high enough to prevent blinding, but low enough to permit transmission of diffuse radiation. The skilled person knows how to select appropriate materials, including, without limitation, fabrics, plastic foils, and the like.

As used herein, the term “illuminating device” relates to any device comprising at least one illuminating light source emitting light of a wavelength comprised in the range of 450 to 495 nm and a diffuser scattering the light of said illuminating light source at least in part in the direction of at least one eye, preferably at least one cornea and/or retina, of a subject. The illuminating device of the present invention is a “portable illuminating device”, thus, preferably, the portable illuminating device is suitable for being carried by a subject. Preferably, a portable illuminating device is an illuminating device being mountable to the body of a subject. Preferably, the portable illuminating device is constructed of components that allow for light-weight construction and, thus, the portable illuminating device can be carried for at least 15 min, at least 30 min, at least 60 min, at least 90 min, at least 2 hours, at least 3 hours, at least 4 hours, at least 5 hours, at least 6 hours, at least 7 hours, or at least 8 hours without inducing discomfort because of its weight. In case the subject is a human, the portable illuminating device, preferably, is mountable in such a way that the portable illuminating device can be carried without using a hand. Preferably, the portable illuminating device is a headdress, more preferably selected from the group consisting of: a hat, a cap, preferably a baseball cap or a peaked cap, and a headband. Also preferably, the portable illuminating device is not a pair of glasses or a pair of goggles.

The term “mounting mechanism” relates to any mechanism capable of keeping the portable illuminating device of the present invention in its intended position. Preferably, the mounting mechanism is a, more preferably, elastic and/or adjustable, band connected to or comprised within the portable illuminating device. The mechanism used to keep the portable illuminating device in place by the mounting mechanism may be direct or indirect. In a direct mounting mechanism, the mounting mechanism interacts with a part of the body of a subject, e.g., preferably, the head or neck of a subject. E.g., in case the portable illuminating device is constructed in the form of a baseball cap, the holding band contacting he back and/or side of the head is the mounting mechanism. In an indirect mounting mechanism, the mounting mechanism interacts with any device, said device itself interacting with a part of the body of a subject. As an example, the portable illuminating device of the present invention may be constructed as a clip-on device including a mounting mechanism which allows for fixing the portable illuminating device of the present invention onto, e.g. a baseball cap or the like. In such case, the fixing device fixing the portable illuminating device onto the cap would be the mounting mechanism. Preferably, the mounting mechanism permits mounting of the portable illuminating device close to at least one eye of a subject, more preferably the mounting mechanism permits mounting of the portable illuminating device on the head, forehead, ear, and/or nose of a subject.

Preferably, the portable illuminating device of the present invention further comprises a blind preventing the light from an illuminating light source comprised in the portable illuminating device from directly illuminating an eye of a subject. It is understood by the skilled person that the blind may be any kind of device preventing the light from an illuminating light source comprised in the portable illuminating device from directly illuminating an eye of a subject, thus, preventing the subject from being glared by the light emitted by the illuminating light source of the portable illuminating device. Thus, preferably, the blind is made from an opaque or light-filtering material and is arranged in a manner such as to intercept the direct way from the illuminating light source to the eye. It is understood by the skilled person that in cases where the diffusor is shaped to at least in part surround the illuminating light source, the diffusor may act as the blind.

Preferably, the portable illuminating device further comprises a sensor for determining if illumination of at least one eye of a subject by said device is advisable. It is understood by the skilled person that the sensor of the present invention may be comprised within the portable illuminating device of the present invention, or may be coupled thereto. It is, however, also envisaged that the sensor is a self-contained device or even an additional functional property of an existing, self-contained device. The skilled person understands that, in cases where the sensor can be physically separated from the portable illuminating device, the sensor and/or the portable illuminating device furthermore comprises at least one interface for data exchange, more particularly a wireless interface, more particularly a radiofrequency coil. Also, preferably, the portable illuminating device and/or sensor comprises at least one driving electronic unit. The driving electronic unit is a unit having at least one data interface for receiving data from the sensor and/or at least one controller interface for regulating the light quality and/or light quantity (light intensity) emitted by the illuminating light source. The driving electronic unit further, preferably, comprises a data processing unit capable of processing data received from the sensor and determining from said data if an increase of the melatonin level in a subject is desirable or not.

Preferably, said sensor comprises means to determine if an increase of the melatonin level in said subject is desirable or not. The term “melatonin level” is understood by the skilled person and relates to the melatonin concentration in said subject, preferably the melatonin concentration in a body fluid, more preferably the melatonin concentration in blood, serum, plasma, or interstitial fluid.

In a preferred embodiment of the present invention, the sensor provides information suitable to determine if an increase of the melatonin level in said subject is desirable. Thus, preferably, the sensor is a sensor of local time and/or longitudinal location. Thus, the sensor preferably is a means of determining the current local time of the location the subject is in; e.g., if the sensor determines that the current local time is 2 p.m., it would be determined that an increase of the melatonin level in said subject is not desirable, and the portable illuminating device would be activated to illuminate the subject with blue and/or green light. In another example, if the sensor determines the current local time is 11 pm, it would be determined that an increase of the melatonin level in said subject is desirable, and the portable illuminating device would be inactivated or would be activated to illuminate the subject with red light. More preferably, the sensor is a means of additionally determining the current longitudinal position of a subject, more preferably a global positioning system (GPS) device. From such information, it can be determined whether an increase of the melatonin level in said subject is desirable at a given time, as described above. Moreover, e.g. in case the sensor determines that the subject moves westward into a new time zone, it can be determined that an increase of the melatonin level in said subject should be delayed to a later point in time to acclimatize the subject to said new time zone, and the portable illuminating device would be activated to illuminate the subject with blue and/or green light. In another preferred embodiment, the sensor comprises an interface, into which the subject can enter data about a planned, preferably future, travel into a different time zone. With this information, the portable illuminating device can calculate optimal illumination cycles, preferably over several days, to accommodate the subject to the new time zone even before travel actually takes place. This is of importance e.g. in cases where jet-lag after arrival in a new time-zone has to be avoided. It is understood that the sensor of time and/or longitudinal location may be a software program obtaining data relating to time, date, and/or location from a handheld device, e.g. a smartphone. It, however, is also envisaged by the present invention that the sensor is a physical device obtaining said data.

In another preferred embodiment, the sensor additionally provides information about the melatonin level in said subject, i.e., preferably, the sensor comprises a melatonin sensor, more preferably a transcutaneous melatonin sensor. Most preferably, the melatonin sensor is a melatonin sensor plaster. The melatonin sensor plaster of the present invention preferably is a plaster for the transcutaneous measurement of melatonin, comprising at least one flexible carrier element having at least one adhesive surface which can be stuck onto a body surface, furthermore comprising at least one sensor radiation source, more particularly a sensor light source, wherein the sensor radiation source is designed to irradiate the body surface with at least one interrogation light, furthermore comprising at least one detector, wherein the detector is designed to detect at least one response light incident from the direction of the body surface. Preferably, the interrogation light and the response light in the sensor plaster are configured such that they are spectrally different. Preferably, the at least one radiation source in the sensor plaster comprises at least one sensor light source comprising an inorganic or organic light-emitting material, more particularly an inorganic or organic light-emitting diode. Preferably, the interrogation light has a wavelength at least partially comprised in the range 240 nm to 330 nm, more preferably the interrogation light has a wavelength at least partially comprised in the range 280 nm to 310 nm, most preferably, the interrogation light has a wavelength of 300 nm. The response light, preferably, has a wavelength at least partially comprised in the range of 300 nm to 500 nm, more preferably a wavelength at least partially comprised in the range 320 nm to 400 nm. Most preferably, the response light has a wavelength at least partially comprised in the range 330 nm to 360 nm. It is understood that, in case the melatonin plaster comprises an excitation filter and/or a response filter, these will be selected to filter light according to the aforesaid specifications. In a preferred embodiment, the melatonin sensor, preferably the transcutaneous melatonin sensor, is incorporated into the portable illuminating device of the present invention. More preferably, the melatonin sensor, preferably the transcutaneous melatonin sensor, is incorporated into the portable illuminating device such that the device fixes the melatonin sensor onto a hair-free area of the skin. Even more preferably, the melatonin sensor, preferably the transcutaneous melatonin sensor, is incorporated into the mounting mechanism of the portable illuminating device, preferably into the rim, the brim, or the headband. Most preferably, the melatonin sensor is incorporated into the mounting mechanism of the portable illuminating device such that the melatonin sensor is pressed onto the forehead of the subject wearing the device. It is understood by the skilled person that in the aforementioned embodiments incorporating the melatonin sensor into the portable illuminating device, the melatonin sensor is preferably comprised in a pocket within the device, preferably the rim, the brim, or the headband, in order to avoid excessive pressure onto the melatonin sensor.

Also preferably, the at least one detector comprises at least one inorganic or organic semiconducting material, more particularly an inorganic or organic photodetector. Also preferably, the sensor comprises at least one interface for data exchange, more particularly a wireless interface, more particularly a radiofrequency coil and/or at least one melatonin sensor driving electronic unit. More preferably, the melatonin sensor driving electronic unit comprises at least one organic component, more particularly an organic conductor track and/or an organic field effect transistor. Most preferably, the melatonin sensor driving electronic unit is designed to control a temporally resolved measurement of the sensor plaster.

Preferably, the sensor plaster is produced in a layer design and has, more preferably, at least two different layer planes. Preferably, the carrier element of the sensor plaster is configured such that it is at least substantially light-tight. More preferably, the adhesive surface of the sensor plaster laterally encloses the detector, wherein, with the sensor plaster stuck onto the body surface, this prevents ambient light from being able to pass to the detector.

In further preferred embodiments, the melatonin sensor, particularly the sensor plaster, further comprises at least one of a data storage device, a filter element, particularly a filter film, and a Fresnel lens.

In a preferred embodiment, the portable illuminating device comprises an oxygen and/or pulse sensor. Oxygen and/or pulse sensors are known in the art. It is understood by the skilled person that changes in melatonin levels in the blood of a subject are often associated with changes in heart rate. Accordingly, an oxygen and/or pulse sensor may also serve as the sole or as an additional sensor for determining if illumination of at least one eye of a subject by said device is advisable, as described herein above. It is further understood by the skilled person that embodiments and modes of incorporation described above for melatonin sensors apply mutatis mutandis to the oxygen and/or pulse sensor of the present invention.

Preferably, the portable illuminating device and/or the sensor further comprise a power source.

Preferably, the power source is a source of electrical energy, more preferably a battery, particularly a polymer battery, an accumulator, a fuel cell and/or a solar panel. It is, however, also envisaged that the power source is an energy harvesting device, e.g. an induction loop. Preferably, the power source is flexible and/or light-weight. Thus, e.g., a Li-accumulator is preferred over a NiCd-accumulator. It is understood by the skilled person that in cases where the sensor is physically separated from the portable illuminating device, the sensor, preferably, also comprises a power source as defined above.

In a preferred embodiment, the portable illuminating device further comprises or is connected to, preferably wirelessly, to a data processing unit comprising tangibly embedded an executable computer code providing at least one of the following functions: i) determining the current local time, comparing said current local time with a wake-up time set by the user, and initiating blue and/or green light illumination if said wake-up time is at most 1 hour, preferably at most 30 min, most preferably at most 15 min after said current local time; ii) determining local time and/or longitudinal location of the portable illuminating device and initiating blue and/or green light illumination if a decrease in melatonin synthesis is desirable, i.e., preferably, if local time is before noon, and/or initiating red light in case an increase in melatonin synthesis is desirable, i.e., preferably, if local time is 8 p.m. or thereafter; iii) receiving instructions from the user relating to a past or future change into a different time zone and adjusting blue and/or green light illumination and/or red light illumination to gradually adapt the user to sleep/wake cycles in said different time zone. It is understood by the skilled person that the aforesaid preferred additional features can, e.g., be established in the form of an executable computer code implemented on a mobile device, e.g. a mobile phone, intercommunicating, preferably wirelessly, with the portable illuminating device.

Advantageously, it was found during the work underlying the present invention that a portable illuminating device is highly comfortable to wear for a subject, especially if it is produced in the form of a headwear comprising a brim, since the surface of the brim can be used as a diffuser, providing an agreeable measure of providing the diffuse illumination required to obtain the degree of illumination to achieve the effects according to the invention without glare. Moreover, it was found that, using the portable illuminating device of the present invention, the time point of onset of melatonin production in a subject can be shifted at will. Also, it was found that by measuring the melatonin concentration in a bodily fluid, initiation of melatonin production can be detected and can be suppressed in cases where said melatonin suppression could potentially be deleterious for the subject, thus obviating the need for continuously carrying the portable illuminating device of the present invention. It will be appreciated that melatonin levels can be monitored at intervals and the subject can be informed, e.g. by an alert triggered by the sensor, putting on the portable illuminating device is advisable. Moreover, by anticipating and/or determining a shift in time zone, the portable illuminating device can be used to pre- and/or readjust the sleep/wake cycle of a subject. It was further found in the work underlying the present invention that cells of the skin can readjust gene expression in response to visible light stimuli, in particular blue light. Since skin cells can also produce melatonin, they can contribute to (re)adjustment of circadian rhythms. Accordingly, the effect of the portable illuminating device can be accomplished or corroborated by illuminating the facial skin, which makes it possible to reduce illumination of the eye and still achieve the same effect. This has the advantages of reducing possible unwanted side-effects (cataracts), and also to reduce potential blinding.

The definitions made above apply mutatis mutandis to the following:

The present invention further relates to the portable illuminating device according to any of the preceding claims for use in preventing and/or treating a disease or disorder caused by or associated with a distorted circadian rhythm. Preferably, the present invention relates to the use of the portable illuminating device according to the present invention in preventing and/or treating a disease or disorder caused by or associated with a distorted circadian rhythm.

In a preferred embodiment, the present invention relates to a method for preventing and/or treating a disease or disorder caused by or associated with a distorted circadian rhythm, comprising illuminating at least one eye and/or at least a part of the facial skin of said subject with light comprising light of a wavelength of 450 to 495 nm and/or with light comprising light of a wavelength of 620 nm to 750 nm, thereby preventing and/or treating a disease or disorder caused by or associated with a distorted circadian rhythm.

It is understood by the skilled person that illumination with blue light is preferred in case a reduction of melatonin levels and/or activation of metabolism is desirable, whereas illumination with red light is preferred in case an increase of melatonin levels and/or relaxation is desired. Accordingly, blue light illumination is preferred in the morning, after sleep, or when cessation of fatigue is desired, whereas red light illumination is preferred in the evening, shortly before sleep, or during sleep.

The term “preventing” refers to retaining health with respect to the diseases or disorders referred to herein for a certain period of time in a subject. It is to be understood that prevention may not be effective in all subjects treated according to the present invention. However, the term requires that a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “treating” refers to ameliorating the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the methods specified herein above.

Preferably, the disease or disorder is caused by or associated with a distorted circadian rhythm. The term “circadian rhythm” is known to the skilled person and relates to any biological process displaying an entrainable endogenous oscillation of about 24 hours. Markers reflecting the circadian state of a mammalian subject are, e.g., melatonin levels, core body temperature, and plasma cortisol levels. Melatonin level is the preferred marker for a subject's circadian state, since it is undetectably low during daytime and rapidly increases in the evening. A distorted circadian rhythm is a circadian rhythm no longer showing the normal 24 h sleep-wake cycle. The most frequent cause for such a distortion exposure of a subject to a disruption in lighting scheme, i.e. exposure of a subject to illumination with light of an intensity and wavelength suited to reset the circadian clock. As a consequence, the sleep-wake cycle is distorted, leading to problems with sleeping during night time, and, conversely, problems to reach optimal mental and/or physical performance during daytime.

The term “disorder”, as used herein, relates to a deviation from normal of a body part, an organ, or a subject sensed by the affected subject as abnormal, preferably sensed as unpleasant, objectionable, and/or painful. Preferably, the disorder is recognizable to others, e.g., a medical practitioner, by signs (symptoms) of said abnormal state. The term “disease” relates to a pathological condition or disorder characterized by an identifiable group of signs or symptoms. Preferred diseases or disorders caused by or associated with a distorted circadian rhythm are Rapid Time Zone Change Syndrome (Jet Lag), Shift Work Sleep Disorder, Delayed Sleep Phase Syndrome, Advanced Sleep Phase Syndrome, or Non 24-Hour Sleep Wake Disorder. It is, however, also envisaged that disorders or diseases not directly related to the sleep-wake cycle, e.g. depression, are prevented or treated according to the present invention.

The present invention also relates to a method of improving physical and/or mental performance of a healthy subject, comprising the step of illuminating at least one eye of said subject with light comprising light of a wavelength of 450 to 495 nm for at least 15 min, thereby improving physical and/or mental performance of a healthy subject. Preferably, the present invention relates to a method of improving physical and/or mental performance of a healthy subject, comprising illuminating at least one eye and/or at least a part of the facial skin of said subject with light comprising light of a wavelength of 450 to 495 nm, thereby improving physical and/or mental performance of a healthy subject.

The method of the present invention, preferably, may comprise steps in addition to those explicitly mentioned above. For example, further steps may relate, e.g., to subjecting said subject to a modified wake-sleep cycle before performing the illuminating step. In a preferred embodiment, the method of improving physical and/or mental performance comprises the further or alternative step of illuminating said subject with light comprising light of a wavelength of 620 nm to 750 nm. It is understood by the skilled person that illumination with blue light is preferred in case a reduction of melatonin levels and/or activation of metabolism is desirable, whereas illumination with red light is preferred in case an increase of melatonin levels and/or relaxation is desired. Accordingly, blue light illumination is preferred in the morning, after sleep, or when cessation of fatigue is desired, whereas red light illumination is preferred in the evening, shortly before sleep, or during sleep. Thus, blue light and red light illumination are, preferably, not administered simultaneously.

The term “healthy subject” relates to a subject not suffering from any of the disorders or diseases caused by or associated with a distorted circadian rhythm as described herein above. More preferably, the term relates to a subject not being afflicted with a diagnosable disease and/or a disorder noticeable to said subject. Most preferably, the term relates to a subject not being afflicted with a disease or a disorder.

As used herein, the term “improving physical and/or mental performance” relates to increasing the ability of a subject to perform mental and/or physical work as determined by a measurable parameter. Preferred parameters are muscular strength, muscular maximal strength, endurance, or alertness as determined by the methods known to the skilled person.

Preferably, the present invention relates to the use of the portable illuminating device according to the present invention in improving physical and/or mental performance of a healthy subject preferably as described herein above.

The preferred duration of illumination in the methods of the present invention is at least 15 min, at least 30 min, at least 60 min, at least 90 min, at least 2 hours, at least 3 hours, at least 4 hours, at least 5 hours, at least 6 hours, at least 7 hours, or at least 8 hours.

Summarizing the findings of the present invention, the following embodiments are preferred:

### Embodiment 1

A portable illuminating device comprising:

i) at least one illuminating light source emitting light of a wavelength comprised in the range of 450 to 495 nm;
ii) a diffuser scattering the light of said illuminating light source at least in part in the direction of at least one eye, preferably at least one cornea and/or retina, of said subject; and
iii) a mounting mechanism for fixing the portable device to the body of a subject.

### Embodiment 2

The portable illuminating device of the preceding embodiment, wherein the mounting mechanism permits mounting the device on the head of the subject.

### Embodiment 3

The portable illuminating device of any of the preceding embodiments, wherein the portable device is a headdress, preferably selected from the group consisting of: a hat, a cap, preferably a baseball cap or a peaked cap, and a headband.

### Embodiment 4

The portable illuminating device of any of the preceding embodiments, wherein the diffuser comprises one of a brim, preferably a curved brim, of a peaked cap, preferably of a baseball cap, of a headband, or a sunblind of a headwear.

### Embodiment 5

The portable illuminating device of any of the preceding embodiments, wherein the diffuser is flexible.

### Embodiment 6

The portable illuminating device of any of the preceding embodiments, wherein the diffuser is adjustable.

### Embodiment 7

The portable illuminating device of any of the preceding embodiments, further comprising a blind preventing the light from said illuminating light source from directly illuminating an eye of said subject.

### Embodiment 8

The portable illuminating device of any of the preceding embodiments, further comprising a power source, preferably one of a battery and an accumulator.

### Embodiment 9

The portable illuminating device of any of the preceding embodiments, wherein the power source is flexible.

### Embodiment 10

The portable illuminating device of any of the preceding embodiments, wherein the illuminating light source comprises a plurality of photon-generating devices having the same or different spectral emission properties.

### Embodiment 11

The portable illuminating device of any of the preceding embodiments, further comprising at least one switch for one of switching on or off the light, changing an intensity of the light, or changing the wavelength of the light.

### Embodiment 12

The portable illuminating device of one of the preceding embodiments, wherein the light emitted by the illuminating light source essentially consists of light of a wavelength comprised in the range of 450 to 495 nm.

### Embodiment 13

The portable illuminating device of any of the preceding embodiments, wherein the illuminating light source further emits light of a wavelength comprised in the range 495-600 nm, or wherein the portable device further comprises a photon-generating device emitting light of a wavelength comprised in the range 495-600 nm.

### Embodiment 14

The portable illuminating device of any of the preceding embodiments, wherein the illuminating light source additionally or, preferably, alternatively emits light of a wavelength comprised in the range 600-800 nm, or wherein the portable device further comprises a photon-generating device emitting light of a wavelength comprised in the range 600-800 nm.

### Embodiment 15

The portable illuminating device of any of the preceding embodiments, wherein the illuminating light source does not emit light of a wavelength of less than 450 nm.

### Embodiment 16

The portable illuminating device of any of the preceding embodiments, wherein the illuminating light source comprises a light-emitting diode.

### Embodiment 17

The portable illuminating device of any of the preceding embodiments, wherein the illuminance of light of a wavelength of 450 to 495 nm on at least one cornea of said subject is at least 100 lux (lm/m²).

### Embodiment 18

The portable illuminating device of any of the preceding embodiments, further comprising a sensor for determining if illumination of at least one eye of a subject by said device is advisable.

### Embodiment 19

The portable illuminating device of the preceding embodiment, wherein the sensor comprises a position determining device for determining the position, preferably the longitudinal position.

### Embodiment 20

The portable illuminating device of any of the preceding embodiments relating to a portable device comprising a sensor, wherein the sensor is a global positioning system (GPS) device.

### Embodiment 21

The portable illuminating device of any of the preceding embodiments relating to a portable device comprising a sensor, wherein the sensor is a transcutaneous melatonin sensor.

### Embodiment 22

The portable illuminating device of the preceding embodiment, wherein the sensor further comprises a melatonin sensor.

### Embodiment 23

The portable illuminating device of the preceding embodiment, wherein the melatonin sensor is a sensor plaster for the transcutaneous measurement of melatonin, comprising at least one flexible carrier element having at least one adhesive surface which can be stuck onto a body surface, furthermore comprising at least one sensor radiation source, more particularly a sensor light source, wherein the sensor radiation source is designed to irradiate the body surface with at least one interrogation light, furthermore comprising at least one detector, wherein the detector is designed to detect at least one response light incident from the direction of the body surface.

### Embodiment 24

The portable illuminating device of any of the preceding embodiments relating to a portable illuminating device comprising a sensor plaster, wherein the interrogation light and the response light in the sensor plaster are configured such that they are spectrally different.

### Embodiment 25

The portable illuminating device of any of the preceding embodiments relating to a portable illuminating device comprising a sensor plaster, wherein the at least one radiation source in the sensor plaster comprises at least one sensor light source comprising an inorganic or organic light-emitting material, more particularly an inorganic or organic light-emitting diode.

### Embodiment 26

The portable illuminating device of any of the preceding embodiments relating to a portable illuminating device comprising a sensor plaster, wherein the sensor plaster comprises at least one detector comprising at least one inorganic or organic semiconducting material, more particularly an inorganic or organic photo detector.

### Embodiment 27

The portable illuminating device of any of the preceding embodiments relating to a portable illuminating device comprising a sensor plaster, wherein the sensor plaster is a self-contained device.

### Embodiment 28

The portable illuminating device of any of the preceding embodiments relating to a portable illuminating device comprising a sensor plaster, wherein the sensor plaster and/or the portable illuminating device furthermore comprises at least one interface for data exchange, more particularly a wireless interface, more particularly a radiofrequency coil.

### Embodiment 29

The portable illuminating device of any of the preceding embodiments relating to a portable illuminating device comprising a sensor plaster wherein the sensor plaster and/or the portable illuminating device furthermore comprises at least one driving electronic unit.

### Embodiment 30

The portable illuminating device of any of the preceding embodiments relating to a portable illuminating device comprising a sensor plaster, wherein the driving electronic unit comprises at least one organic component, more particularly an organic conductor track and/or an organic field effect transistor.

### Embodiment 31

The portable illuminating device of any of the preceding embodiments relating to a portable illuminating device comprising a sensor plaster, wherein the driving electronic unit is designed to control a temporally resolved measurement of the sensor plaster.

### Embodiment 32

The portable illuminating device of any of the preceding embodiments relating to a portable illuminating device comprising a sensor plaster, wherein the sensor plaster furthermore comprises at least one energy generating device for providing electrical energy, more particular a solar cell or an induction loop.

### Embodiment 33

The portable illuminating device of any of the preceding embodiments relating to a portable illuminating device comprising a sensor plaster, wherein the sensor plaster furthermore comprises at least one data storage device.

### Embodiment 34

The portable illuminating device of any of the preceding embodiments relating to a portable illuminating device comprising a sensor plaster, wherein the sensor plaster furthermore comprises at least one filter element, more particularly at least one filter film.

### Embodiment 35

The portable illuminating device of any of the preceding embodiments relating to a portable illuminating device comprising a sensor plaster wherein the sensor plaster furthermore comprises at least one imaging system, more particularly at least one Fresnel lens.

### Embodiment 36

The portable illuminating device of any of the preceding embodiments relating to a portable illuminating device comprising a sensor plaster, wherein the sensor plaster furthermore comprises at least one electrical energy storage device, more particularly at least one polymer battery.

### Embodiment 37

The portable illuminating device of any of the preceding embodiments relating to a portable illuminating device comprising a sensor plaster, wherein the carrier element of the sensor plaster is configured such that it is at least substantially light-tight.

### Embodiment 38

The portable illuminating device of any of the preceding embodiments relating to a portable illuminating device comprising a sensor plaster, wherein the adhesive surface of the sensor plaster laterally encloses the detector, wherein, with the sensor plaster stuck onto the body surface, this prevents ambient light from being able to pass to the detector.

### Embodiment 39

The portable illuminating device of any of the preceding embodiments relating to a portable illuminating device comprising a sensor plaster, wherein the sensor plaster is produced in a layer design and has at least two different layer planes, preferably manufactured by print technology.

### Embodiment 40

The portable illuminating device of any of the preceding embodiments relating to a portable illuminating device comprising a melatonin sensor, wherein the melatonin sensor, preferably the transcutaneous melatonin sensor, is incorporated into the portable illuminating device such that the device fixes the melatonin sensor onto a hair-free area of the skin.

### Embodiment 41

The portable illuminating device of any of the preceding embodiments relating to a portable illuminating device comprising a melatonin sensor, wherein the melatonin sensor is incorporated into the mounting mechanism of the portable illuminating device such that the melatonin sensor is pressed onto the forehead of the subject wearing the portable illuminating device.

### Embodiment 42

The portable illuminating device of any of the preceding embodiments, wherein the diffuser scatters light emitted by the illuminating light source at least in part in the direction of at least one eye and in the direction of at least a part of the facial skin of a subject.

### Embodiment 43

The portable illuminating device of any of the preceding embodiments, wherein the diffuser scatters light emitted by the illuminating light source such that at least 20% of the facial skin of said subject are illuminated.

### Embodiment 44

The portable illuminating device of the preceding embodiment, wherein said 20% of the facial skin are illuminated with an average illuminance of at least 100 lux (lm/m²).

### Embodiment 45

The portable illuminating device of any of the preceding embodiments, wherein said diffuser scatters light emitted by the illuminating light source such that at most 20% of the light scattered at least in part in the direction of at least one eye and in the direction of at least a part of the facial skin of the subject illuminates a retina or the retinas of said subject.

### Embodiment 46

A portable illuminating device according to any of the preceding embodiments for use in preventing and/or treating a disease or disorder caused by or associated with a distorted circadian rhythm.

### Embodiment 47

The portable illuminating device for use according to the preceding embodiment, wherein the disease or disorder caused by or associated with a distorted circadian rhythm is Rapid Time Zone Change Syndrome (Jet Lag), Shift Work Sleep Disorder, Delayed Sleep Phase Syndrome, Advanced Sleep Phase Syndrome, or Non 24-Hour Sleep Wake Disorder.

### Embodiment 48

A method of improving physical and/or mental performance of a healthy subject, comprising the step of illuminating at least one retina of said subject with light comprising light of a wavelength of 450 to 495 nm for at least 15 min, thereby improving physical and/or mental performance of a healthy subject.

### Embodiment 49

The method of improving physical and/or mental performance according to the preceding embodiment, wherein the light comprising light of a wavelength of 450 to 495 nm is provided by the portable device according to one of the preceding embodiments.

### Embodiment 50

The method of improving physical and/or mental performance according to any of the preceding embodiments, wherein the illuminating is performed on at least one cornea of said subject with an illuminance of light of a wavelength of 450 to 495 nm of at least 100 lux (lm/m²).

### Embodiment 51

The method of improving physical and/or mental performance according to any of the preceding embodiments, wherein the illuminating is performed for at least 30 min, at least 60 min, or at least 90 min, at least 2 hours, at least 3 hours, at least 4 hours, at least 5 hours, at least 6 hours, at least 7 hours, or at least 8 hours.

### Embodiment 52

The method of improving physical and/or mental performance according to any of the preceding embodiments, wherein the illuminating is using a portable device according to any of the preceding embodiments.

### Embodiment 53

Method of improving physical and/or mental performance of a healthy subject, comprising illuminating at least one eye and/or at least a part of the facial skin of said subject with light comprising light of a wavelength of 450 nm to 495 nm and/or illuminating said subject with light comprising light of a wavelength of 620 nm to 750 nm, thereby improving physical and/or mental performance of a healthy subject.

### Embodiment 54

Method for preventing and/or treating a disease or disorder caused by or associated with a distorted circadian rhythm, comprising illuminating at least one eye and/or at least a part of the facial skin of said subject with light comprising light of a wavelength of 450 to 495 nm and/or with light comprising light of a wavelength of 620 nm to 750 nm, thereby preventing and/or treating a disease or disorder caused by or associated with a distorted circadian rhythm.

### Embodiment 55

Use of the portable illuminating device (110) according to any of the preceding embodiments relating to a portable illuminating device in preventing and/or treating a disease or disorder caused by or associated with a distorted circadian rhythm.

### Embodiment 56

Use of the portable illuminating device according to any of the preceding embodiments relating to a portable illuminating device in improving physical and/or mental performance of a healthy subject.

All references cited in this specification are herewith incorporated by reference with respect to their entire disclosure content and the disclosure content specifically mentioned in this specification.

Further optional features and embodiments of the invention will be disclosed in more detail in the subsequent description of preferred embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

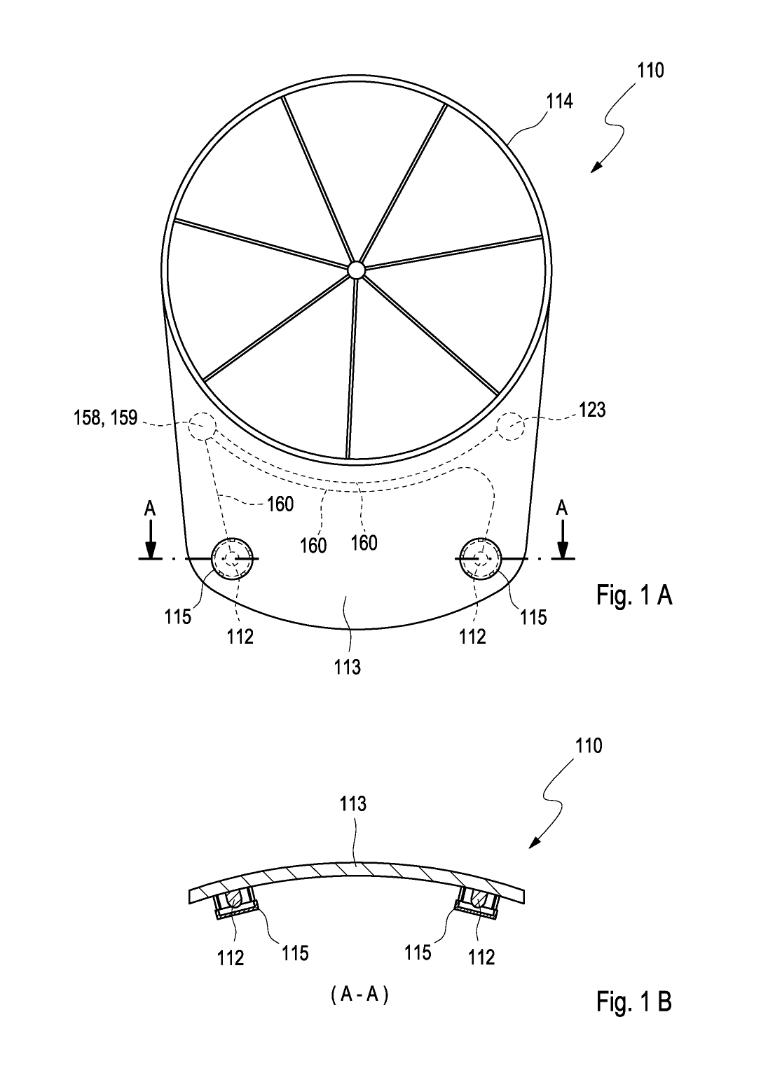

FIGS. 1A and 1B show an exemplary embodiment of a portable illuminating device in a bottom view (FIG. 1A) and in a cross-sectional view along cutting line A-A in FIG. 1A (FIG. 1B);

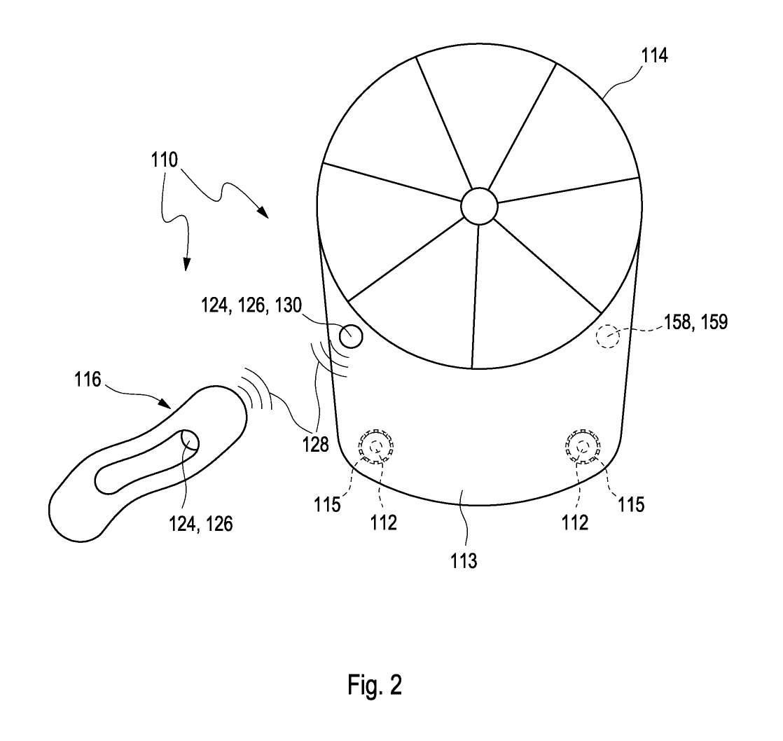

FIG. 2 shows an exemplary embodiment of a portable illuminating device according to the invention, comprising a physically separated sensor plaster;

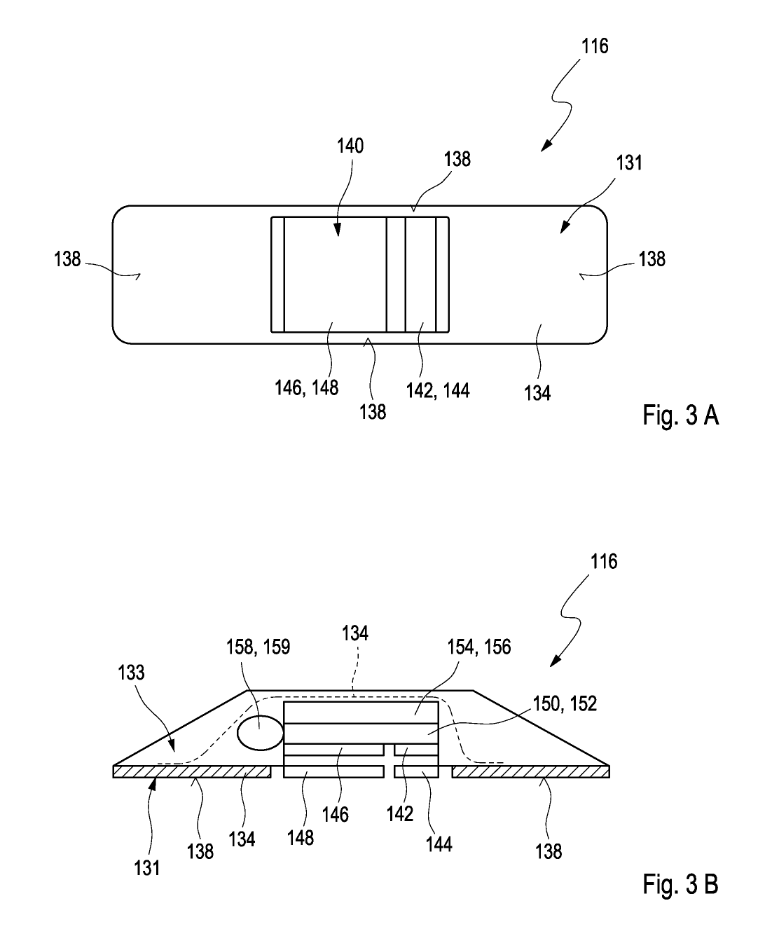

FIGS. 3A and 3B show an exemplary embodiment of a sensor plaster according to the invention in a top view (FIG. 3A) and in a cross-sectional view (FIG. 3B).

### EXAMPLE 1

An exemplary embodiment of a portable illuminating device 110 according to the invention is illustrated highly schematically in FIGS. 1A and 1B. In this exemplary embodiment, the portable illuminating device 110 is shaped as a baseball cap, comprising two illuminating light sources 112, a diffuser 113, which is the part of the shield (also denoted as bill, brim, or peak) of said baseball cap facing the body of the carrier, and a mounting mechanism 114, which is the fixing band of said baseball cap. The exemplary embodiment further comprises a blind 115 for each of the illuminating light sources 112, a sensor 123 determining the current local time, and an electrical energy source 158, which may be a or may be coupled to an energy generating device 159. The illuminating light sources 112, the electrical energy source 158, and the sensor 123 are connected by an electrical connection 160.

### EXAMPLE 2

A further exemplary embodiment of a portable illuminating device 110 according to the invention is illustrated highly schematically in FIG. 2. In this exemplary embodiment, the portable illuminating device 110 comprises two physically separate entities, i.e. an illuminating part as described in Example 1, plus a sensor plaster 116 for transcutaneous measurement of the melatonin concentration. The two parts of the portable illuminating device 110 communicate via wireless communication 128, thus the sensor plaster 116 and the illuminating part as described in Example 1 both comprise an interface 124 for wireless communication, preferably a radio frequency interface 126. Further comprised is a driving electronic unit 130 receiving data from the sensor plaster 116 and regulating the light intensity emitted by the illuminating light source 112.

### EXAMPLE 3

A further exemplary embodiment of a sensor plaster 116 is schematically illustrated in FIGS. 3A and 3B in different viewing directions. The sensor plaster 116 has a front side 131, which, in a state in which the sensor plaster 116 has been applied to a body surface (not illustrated in the figures), faces the body surface, and a rear side 133 facing away from the body surface. In this case, FIG. 3A shows a plan view of the front side 131 of the sensor plaster 116, whereas FIG. 3B shows a perspective view of the sensor plaster 116 highly schematically. In this perspective view, however, a layer construction is indicated symbolically, in a departure from the perspective illustration. The front side 131 is at the bottom in the illustration in accordance with FIG. 2B.

As emerges from the plan view of the front side 131 of the sensor plaster 116 in accordance with FIG. 3A, the sensor plaster 116 comprises a flexible carrier element 134. Said flexible carrier element 134 can be configured in light-tight fashion, for example, and can serve as a carrier for the other elements of the sensor plaster 116. By way of example, the flexible carrier element 134 can be configured in the form of a rectangular, elongate strip and can comprise for example a carrier material comprising at least one flexible material and/or a layer construction of such flexible materials. By way of example, it is possible here to use plastic materials, ceramic materials, paper materials, glass materials or combinations of the aforementioned and/or other materials.

The carrier element 134 is intended to be configured flexibly in such a way that it can be deformed in such a way that an adaptation to the respective body surface on which the measurement is intended to take place is possible. In this respect, the term “flexible” should be interpreted as “deformable” in the context of the present invention. As indicated by the dashed line in FIG. 3B, the carrier element 134 can completely cover the other elements of the sensor plaster 116 on the rear side 133.

The sensor plaster 116 has at least one light source 142, one or a plurality of detector areas of at least one detector 146, one or a plurality of filters 144, 148, optical elements, protective elements or other components.

The carrier element 134 has an adhesive surface 138 in the exemplary embodiment in accordance with FIG. 3A. The adhesive surface 138 can be configured as a self-adhesive adhesive surface 138 by means of an adhesive, for example. In particular, said adhesive surface 138 can in turn be configured in such a way that, when the sensor plaster 116 has been stuck in place, no ambient light can pass to the detector 146.

In the exemplary embodiment illustrated, the sensor plaster 116 has an optical unit 140 as a bottommost—as viewed from the front side 131—element of a layer construction. In the exemplary embodiment illustrated, said optical unit 140, the layer construction of which can be discerned in FIG. 3B, for example, comprises a light source 142, which is configured as a light emitting diode (LED), preferably an organic light-emitting diode (OLED), for example. An excitation filter 144, for example a filter film, can be applied on said light source 142, such that said excitation filter 144 faces toward the body surface.

In the exemplary embodiment illustrated, the optical unit 140 furthermore comprises a detector 146, for example an organic solar cell. Said detector 146 is provided, for example, with a response filter 148, for example once again in the form of a filter film adhesively bonded onto the detector 146.

In the next layer plane, on that side which faces away from the body surface, the sensor plaster 116 in the exemplary embodiment illustrated comprises an electronic unit 150. The layer construction illustrated can be realized particularly simply in terms of printing technology, for example, and brings about short electronic transmission paths and also a flat and compact design. The electronic unit 150 can comprise for example a driving electronic unit 152 for the driving and/or evaluation of the data measured by the other components of the sensor plaster 116. By way of example, by means of this driving electronic unit 152, the light source 142 can be excited to emit interrogation light and/or the detector 146 can be excited to detect response light. Furthermore, the driving electronic unit 152 can also comprise one or a plurality of data storage devices in order to perform at least buffer-storage of the measurement results that were obtained by means of the detector 146. Various other configurations are possible.

Furthermore, the sensor plaster 116 in accordance with the exemplary embodiment illustrated in FIGS. 3A and 3B comprises a communication unit 154. Said communication unit 154 can be configured using RFID technology, for example, and/or can comprise a radiofrequency coil in order to realize the wireless as designated symbolically by reference numeral 128 in FIG. 2. The communication unit 154, too, can be driven wholly or partly by the driving electronic unit 152 and/or can have a separate driving electronic unit 152.

Furthermore, the sensor plaster 116 in the exemplary embodiment illustrated in FIG. 3B comprises an electrical energy source 158. While the communication unit 154, the electronic unit 150 and the other elements of the sensor plaster 116 are arranged one above another in a layer design in the exemplary embodiment illustrated in FIGS. 3A and 3B, which, however, likewise need not necessarily be the case, the electrical energy source 158 is arranged alongside this layer construction in FIG. 3B. Alternatively or additionally, however, the at least one electrical energy source 158 can also be integrated fully or partly into the layer construction of the units.

The electrical energy source 158 can comprise for example a printed battery, for example a printed polymer battery, or an induction loop. As an alternative or in addition to the at least one electrical energy source 158, the sensor plaster 116 can also comprise one or a plurality of energy generating devices, which are designated symbolically by the reference numeral 159 in FIG. 3B. Said energy generating devices 159 can, as indicated symbolically in FIG. 3B, be configured jointly with the electrical energy source 158, but can also be embodied wholly or partly spatially separately from said electrical energy source 158.

By way of example, the required electrical energy can be radiated in externally, in the manner used in conventional transponder technology. For this purpose, by way of example, the communication unit 154 can receive its energy required from the incident electromagnetic waves. Alternatively or additionally, the energy generating device 159 can also comprise for example one or a plurality of solar cells, for example once again one or a plurality of organic solar cells. This at least one solar cell can then comprise for example at least one solar cell area which faces the rear side 133 of the sensor plaster 116 and which is preferably at least not completely covered by the carrier element 134, such that incidence of ambient light, more particularly sunlight, onto said solar cell area is possible. Once again alternatively or additionally, the energy generating device 159 can comprise one or a plurality of thermoelectric converters, for example one or a plurality of Peltier or Seebeck elements. Other configurations are also possible, or else combinations of the aforementioned and/or other possibilities for the configuration of the energy generating device 159.

### LIST OF REFERENCE SYMBOLS

110 Portable illuminating device
112 Illuminating light source
113 Diffuser
114 Mounting mechanism
115 Blind
116 Sensor plaster
123 Sensor
124 Interface
126 Radio frequency interface
128 Wireless communication
130 Driving electronic unit (Portable illuminating device)
131 Front side
133 Rear side
134 Carrier element
138 Adhesive surface
142 Sensor Light source
144 Excitation filter
146 Detector
148 Response filter
150 Electronic unit
152 Driving electronic unit (Sensor plaster)
154 Communication unit
156 Interface
158 Electrical energy source
159 Energy generating device
160 Electrical connection

1-18. (canceled)

19. A portable illuminating device (110) comprising:
i) at least one illuminating light source (112) emitting light of a wavelength comprised in the range of 450 to 495 nm;
ii) a mounting mechanism (114) for fixing the portable illuminating device (110) to the body of a subject; and
iii) a diffuser (113) scattering the light of said illuminating light source (112) at least in part in the direction of at least one eye, preferably at least one cornea and/or retina, of said subject.

20. The portable illuminating device (110) of claim 19, wherein the portable illuminating device (110) is a headdress, preferably selected from the group consisting of: a hat, a cap, preferably a baseball cap or a peaked cap, and a headband.

21. The portable illuminating device (110) of claim 19, wherein the diffuser (113) comprises one of a brim, preferably a curved brim, of a peaked cap, preferably of a baseball cap, of a headband, or a sunblind of a headwear.

22. The portable illuminating device (110) of claim 19, further comprising a blind (115) preventing the light from said illuminating light source from directly illuminating an eye of said subject.

23. The portable illuminating device (110) of claim 19, wherein the illuminating light source (112) comprises a plurality of photon-generating devices having the same or different spectral emission properties.

24. The portable illuminating device (110) of claim 19, wherein the illuminating light source (112) further emits light of a wavelength comprised in the range 495-600 nm and/or 600-800 nm, or wherein the portable illuminating device (110) further comprises a photon-generating device emitting light of a wavelength comprised in the range 495-600 nm and/or a photon-generating device emitting light of a wavelength comprised in the range 600-800 nm.

25. The portable illuminating device (110) of claim 19, wherein the illuminance of light of a wavelength of 450 to 495 nm on at least one cornea of said subject is at least 100 lux (lm/m2).

26. The portable illuminating device (110) of claim 19, further comprising a sensor (116; 123) for determining if illumination of at least one eye of a subject by said device is advisable.

27. The portable illuminating device (110) of claim 19, wherein the sensor (116; 123) comprises a position determining device for determining the position, preferably the longitudinal position.

28. The portable illuminating device (110) of claim 19, wherein the sensor (116; 123) further comprises a melatonin sensor (116).

29. The portable illuminating device (110) of claim 19, wherein the diffuser (113) scatters light emitted by the illuminating light source at least in part in the direction of at least one eye and in the direction of at least a part of the facial skin of a subject.

30. The portable illuminating device (110) of claim 19, wherein the diffuser (113) scatters light emitted by the illuminating light source such that at least 20% of the facial skin of said subject are illuminated.

31. The portable illuminating device (110) of claim 29, wherein said 20% of the facial skin are illuminated with an average illuminance of at least 100 lux (lm/m2).

32. The portable illuminating device (110) of claim 19, wherein said diffuser (113) scatters light emitted by the illuminating light source such that at most 20% of the light scattered in the direction of at least one eye and in the direction of at least a part of the facial skin of the subject illuminates a retina or the retinas of said subject.

33. A method of improving physical and/or mental performance of a healthy subject, comprising illuminating at least one eye and/or at least a part of the facial skin of said subject with light comprising light of a wavelength of 450 nm to 495 nm and/or illuminating said subject with light comprising light of a wavelength of 620 nm to 750 nm, thereby improving physical and/or mental performance of a healthy subject.

34. The method of claim 32, wherein the illumination is performed using a portable device comprising a diffuser.

35. The method of claim 33, wherein the diffuser comprised in said portable device scatters light emitted by the illuminating light source such that at least 20% of the facial skin of said subject are illuminated.

36. The method of claim 34, wherein said at least 20% of the facial skin are illuminated with an average illuminance of at least 100 lux (lm/m2).

37. The method of claim 32, comprising the step of illuminating at least one retina of said subject with light comprising light of a wavelength of 450 to 495 nm for at least 15 min.

38. A method for preventing and/or treating a disease or disorder caused by or associated with a distorted circadian rhythm in a subject, comprising illuminating at least one eye and/or at least a part of the facial skin of said subject with light comprising light of a wavelength of 450 to 495 nm and/or with light comprising light of a wavelength of 620 nm to 750 nm, thereby preventing and/or treating a disease or disorder caused by or associated with a distorted circadian rhythm.
